(19) United States
(12) Patent Application Publication
(10) Pub. No.: US 2013/0197002 A1
(43) Pub. Date: Aug. 1, 2013

(54) BRIMONIDINE FOR TREATING VISUAL DISORDERS MEDIATED BY CENTRAL VISUAL PROJECTIONS FROM THE EYE

(71) Applicant: Allergan, Inc., Irvine, CA (US)

(72) Inventors: Ursula Staubli, Laguna Beach, CA (US); Alan C. Foster, San Diego, CA (US); Daniel W. Gil, Corona Del Mar, CA (US); John E. Donello, Dana Point, CA (US)

(73) Assignee: ALLERGAN, INC., Irvine, CA (US)

(21) Appl. No.: 13/752,871

(22) Filed: Jan. 29, 2013

Related U.S. Application Data
(60) Provisional application No. 61/592,115, filed on Jan. 30, 2012.

(51) Int. Cl. A61K 31/498

(52) U.S. Cl. CPC A61K 31/498
USPC 514/249

(57) ABSTRACT

The present invention relates to a method for treating visual disorders mediated by lateral geniculate nucleus, superior colliculus and the visual cortex by administering to a patient in need of such treatment, compounds acting at the alpha 2 adrenergic receptor.

### RELATED APPLICATION

This application claims the benefit of U.S. Provisional Application Ser. No. 61/592,115, filed Jan. 30, 2012, which is incorporated herein by reference in its entirety.

### FIELD OF THE INVENTION

The present invention relates to a method for treating visual disorders mediated by lateral geniculate nucleus, superior colliculus and the visual cortex by administering to a patient in need of such treatment (5-bromo-quinoxalin-6-yl)-imidazolidin-2-ylidene-amine acting at the alpha 2 adrenergic receptor.

### SUMMARY OF THE RELATED ART

The compound (5-bromo-quinoxalin-6-yl)-imidazolidin-2-ylidene-amine (structure shown below) is generically known as brimonidine tartrate and is sold under the trademark ALPHAGAN®P (available from Allergan, Inc.).

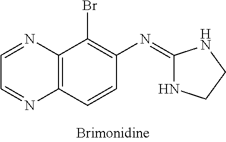 [SMILES: Brc1c(N=C2NCCN2)ccc2nccnc12]

Pharmacological activation of the alpha 2 adrenergic receptor by brimonidine is a well established treatment for various visual disorders of the eye. Alpha 2 adrenergic agonists, such as brimonidine, have physiological effects beyond the eye in the central nervous system where they interact with the adrenergic central pathways. Thus, alpha 2 adrenergic agonists might also be beneficial for treating visual system disorders mediated by central visual areas, including, but not limited to the visual cortex.

The visual cortex is one synapse removed from the eye and integrates visual signals generated by the retina. It is thus essential for decoding, processing and transforming visual inputs originating in the eye, and proper visual cortical function is necessary for normal vision. Noradrenaline released from the nerve terminals in visual cortex gates experience dependent modification of visual responsiveness including ocular dominance shifts after monocular deprivation (Imamura et al., Neuroscience 147 (2007) 508-521).

The effect of bromonidine was investigated in the visual cortex using brain slices prepared from primary visual cortex to determine possible drug interactions with visual cortex plasticity mechanisms, in particular long-term potentiation (LTP). LTP serves as a cellular model for visual cortex plasticity and has functional consequences on visual evoked responses (Cooke and Bear, The Journal of Neuroscience, Dec. 1, 2010, 30(48):16304-16313).

### BRIEF DESCRIPTION OF THE DRAWINGS

FIG. 1 shows dose-dependent facilitation of LTP in rat visual cortex by brimonidine and its reversal by alpha 2 pan-antagonist atipamezole.

FIG. 2 shows that brimonidine counteracts the loss in signal power in Long Evans rats eight to nine weeks after a 1 sec optic nerve crush (ONC) treatment.

FIG. 3 shows that brimonidine restores the contrast sensitivity in treated Long Evans rats 22-23 weeks after blue-light treatment.

### SUMMARY OF THE INVENTION

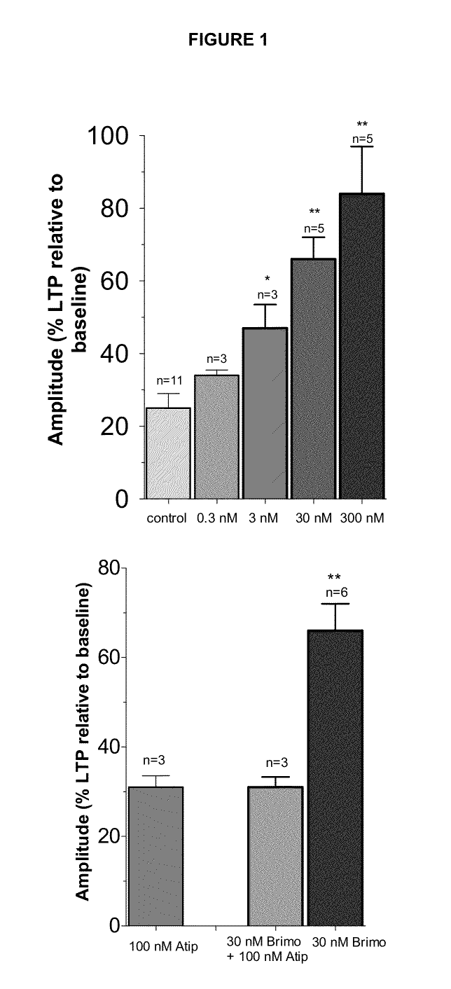

We have discovered that administering brimonidine to visual cortex slices was found to produce a marked and dose-dependent enhancement of LTP, with a threshold dose of 3 nM (FIG. 1, left panel), an effect that was fully suppressed in presence of the alpha 2 pan antagonist atipamezole (FIG. 1, right panel). Atipamezole is a potent, selective and specific antagonist of both centrally and peripherally located alpha 2-adrenoreceptors (Virtanen R, Savola J M, Saano V, Arch Int. Pharmacodyn. Ther. 297: 190-204, 1989).

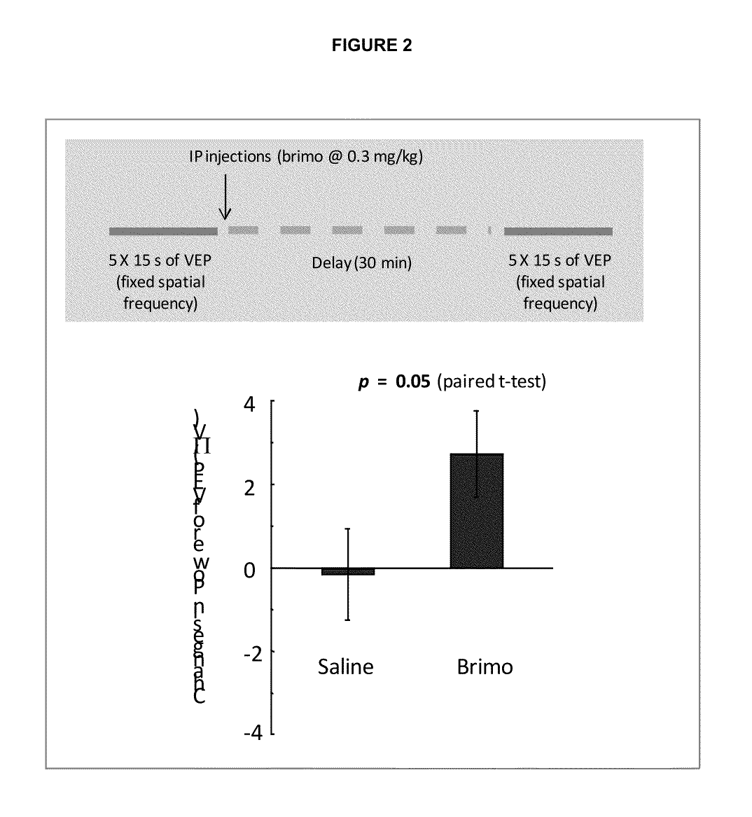

FIG. 1 shows Brimonidine given 8 weeks after ONC acutely reduced the loss in sweep VEP amplitude measured in visual cortex. Next brimonidine was tested in two different animal models to assess its ability to reverse visual dysfunction, as might be predicted from the above described in vitro functional profile. First brimonidine was tested in a rat model of glaucoma consisting of a 1 sec unilateral optic nerve crush (ONC), a treatment that partially destroys retinal ganglion cells including their axonal projections to visual cortex. Following ONC, rats were found to exhibit a significant loss in visual acuity as measured by sweep VEP methodology. However, when administered eight weeks after ONC via systemic route, brimonidine partially restored the power of the sVEP amplitude (see FIG. 2), in agreement with the suggested therapeutic potential of alpha 2 adrenergic activation for visual dysfunction.

FIG. 2 shows that brimonidine counteracts the loss in signal power in Long Evans rats eight to nine weeks after a 1 sec ONC treatment. Saline was used as a control. Half of the rats were injected with 0.3 mg/kg brimonidine and the other half with saline in the first test; cross-over exposure took place one week later; the spatial frequencies were fixed at 0.2 cpd. The left and middle graphs show data from individual rats collected 30 min before and after saline or brimonidine injection. The right graph compares individual changes (delta) in power under control vs drug condition. Thus, brimonidine reduced the deficit in visual performance in this model of visual system degeneration.

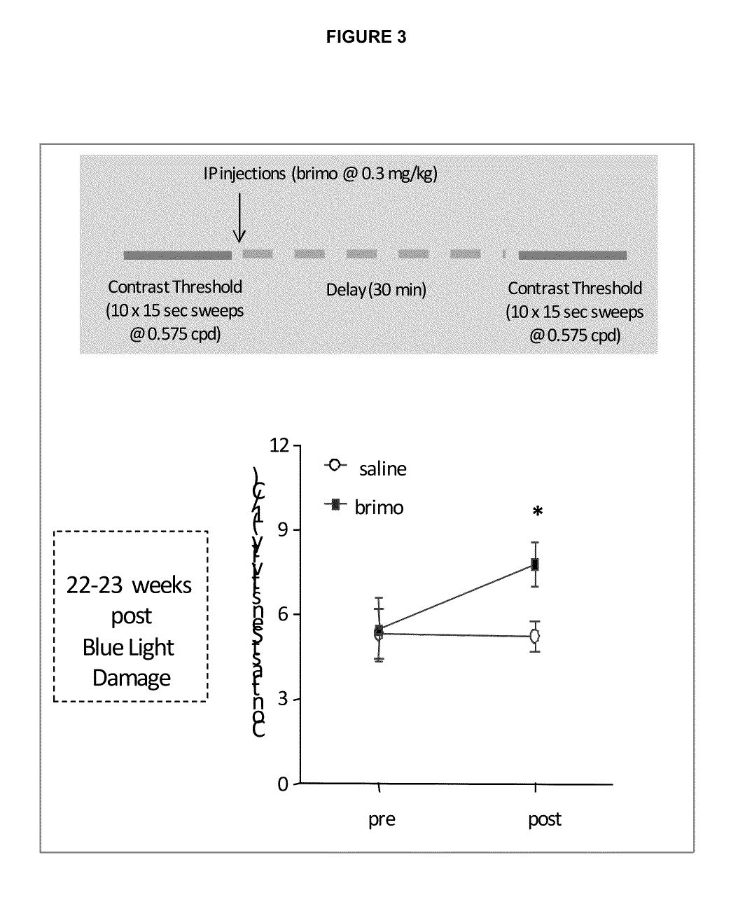

The ability of brimonidine to restore visual dysfunction was further confirmed in the blue light model. Blue-light treatment damages photoreceptors in the retina, and has been proposed as a model of age-related macular degeneration (ARMD; Wielgus et al., Photochem. Photobiol. Sci., 2010, 9, 1505-1512). In blue-light treated Long-Evans rats, contrast sensitivity, an important measure of visual performance, was significantly impaired, a deficit that was partially restored by acute brimonidine administration (FIG. 3).

FIG. 3 shows that brimonidine restores the contrast sensitivity in rats 22-23 weeks after blue-light treatment. Saline was used as a control. Half of the rats were injected with 0.3 mg/kg brimonidine and the other half with saline in the first test; cross-over exposure took place one week later; the spatial frequencies were fixed at 0.575 cpd. The left and middle graphs show data from individual rats collected 30 min before and after saline or brimonidine injection. The right graph illustrates the mean values of contrast sensitivities shown in left and middle graph. The open bar indicates the contrast sensitivity measured in control rats using the same spatial frequency. Thus, brimonidine half-way normalized the deficit in visual performance in this model of visual degenerative disease.

These findings provide evidence that activation of alpha 2 adrenergic receptors by brimonidine is very effective at improving cortical synaptic plasticity, a strategy predicted to have therapeutic benefits in disorders where central visual plasticity needs to be restored or increased. A prime example of a visual disorder mediated by visual cortex is amblyopia. Amblyopia is defined as poor or indistinct vision by an eye that is physically normal. Amblyopia can be initiated by poor transmission of the visual image to the visual cortex during childhood. Abnormal visual processing may be caused by a form of deprivation (i.e. cataracts), anisometropia (different retinal image size, or magnification, in each eye), or suppression resulting from strabismus (misalignment of the eyes). A prolonged transmission of poor quality visual images induces a physiological change within the visual cortex that alters the perception within the visual cortex. Briefly, the visual cortex will “ignore” the poor vision from one eye. Hence amblyopes often lack visual acuity and stereopsis.

Visual system disorders which may be treated with alpha 2 adrenergic agonists include macular edema, dry and wet macular degeneration, choroidal neovascularization, geographic atrophy, optic neuritis, rod dystrophies, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, retinitis pigmentosa, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigement epitheliitis, retinitis pigmentosa, proliferative vitreal retinopathy (PVR), age-related macular degeneration (ARMD), diabetic retinopathy, diabetic macular edema, retinal detachment, retinal tear, uveitus, cytomegalovirus retinitis, glaucoma, amblyopia, stroke-induced blindness, visual system disorder in Parkinson's disease, Alzheimer's disease and multiple sclerosis, seizure-induced cortical blindness, induced visual system disorder, epileptic blindness, multiple sclerosis (MS)-induced visual system disorder, and congenital and childhood myotonic dystrophy type 1-induced visual system disorder.

The term “pharmaceutically acceptable salts” according to the invention include therapeutically active, non-toxic base or acid salt forms, which compound (5-bromo-quinoxalin-6-yl)-imidazolidin-2-ylidene-amine is able to form.

The acid addition salt form of (5-bromo-quinoxalin-6-yl)-imidazolidin-2-ylidene-amine that occurs in its free form as a base, can be obtained by treating the free base with an appropriate acid such as an inorganic acid, for example hydrochloric acid, hydrobromic acid, sulfuric acid, phosphoric acid, nitric acid and the like; or an organic acid such as for example, acetic, hydroxyacetic, propanoic, lactic, pyruvic, malonic, fumaric acid, maleic acid, oxalic acid, tartaric acid, succinic acid, malic acid, ascorbic acid, benzoic acid, tannic acid, pamoic acid, citric, methylsulfonic, ethanesulfonic, benzenesulfonic, formic and the like (Handbook of Pharmaceutical Salts, P. Heinrich Stahal & Camille G. Wermuth (Eds), Verlag Helvetica Chemica Acta-Zürich, 2002, 329-345).

The activation of alpha 2 adrenergic receptors by brimonidine confirms that alpha 2 adrenergic receptors are effective at enhancing cortical synaptic plasticity, and have therapeutic benefits in disorders where central visual plasticity needs to be restored or increased.

Alpha 2 adrenergic agonists may be administered at pharmaceutically effective amounts. Such amounts are normally the minimum dose necessary to achieve the desired therapeutic effect. The actual amount of the compound to be administered in any given case will be determined by a physician taking into account the relevant circumstances. In one embodiment, the compounds of the invention are administered at doses that are pharmaceutically effective but that do not cause sedation. The patient may be given the compounds of the invention orally or by local delivery to the eye. Local delivery includes topical delivery, in which an ophthalmologically acceptable formulation is instilled in the eye via an eye dropper or other applicator, delivery by injection into the eye, or delivery by a drug delivery system that is implanted into the eye or eye lid and releases drug over a period of time. Ophthalmic formulations of drug products are well known in the art and described in, for example, U.S. Patent Application Publication No. 20050059583; No. 20050277584; U.S. Pat. No. 7,297,679; and No. 20070015691; and U.S. Pat. Nos. 5,474,979 and 6,582,718. Drug delivery systems are also known in the art and described in, for example, U.S. Pat. 7,931,909.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

### General Procedure Followed in Obtaining Experimental Data

Long-Term Potentiation in Visual Cortex Slice

Following decapitation of the anesthetized rat, the brain was rapidly removed and immersed in ice-cold artificial cerebrospinal fluid (ACSF) containing (in mM) NaCl 124, KCl 3, KH₂PO₄ 1.25, CaCl₂ 3.4, MgSO₄ 2.5, NaHCO₃ 26, and D-glucose 10. A block of visual cortex was created by removing the frontal ⅔ portion of the brain and the cerebellum. Coronal visual cortex slices of 350 μm thick were prepared from young adult (200-300 g) male Sprague-Dawley rats using a vibratome (VT 1000 S; Leica). The slices were maintained in an interface recording chamber perfused with preheated ACSF. Slices were continuously perfused with this solution at a rate of 1.00-1.50 ml/min while the surface of the slices was exposed to warm, humidified 95% O₂/5% CO₂ and maintained at 31±1° C. Visual cortex slices were allowed to recover for 1 hr before recording began. A single stimulating and recording electrode was placed in layer IV and III, respectively, to generate and record field excitatory postsynaptic potentials (fEPSPs). Pulses were administered at 0.05 Hz using a current that produced a fEPSP that is 50% of the maximum spike free response. An input-output (IO) curve is done to determine the stimulation needed to achieve a stable baseline. Following a 15 min stable baseline recording period, a train of 5 theta bursts (each burst containing four pulses at 100 Hz with an inter-burst interval of 200 ms) is delivered to the slice. This is repeated 2 additional times with a 1 minute inter-train interval, and the level of LTP was recorded for at least 30 min. Changes in amplitude of the synaptic response were used to measure the extent of LTP, since the amplitude was determined to be the more consistent parameter than the slope of the response. Control LTP values were obtained from slices not treated with drug. Different slices were used to study drug effects on LTP. Brimonidine was infused after 15 min baseline recording for duration of 20 minutes followed by LTP induction. Drug washout began 5 minutes after tetanization. Recording of the amplitude before, during, and after drug infusion was continuously done at 0.05 Hz. Slices involving suppression of LTP facilitation used the same basic protocol but were exposed to atipamazole 10 min prior to combined infusion of atipamazole and brimonidine for 15 min, followed by LTP induction and washout 5 min after tetanus. LTP was recorded for at least 30 min after induction.

Sweep VEP Recording in Awake Rat

Sweep VEP recordings were performed in awake rats sitting in a restrainer, and responses were measured via an electrode permanently implanted in layer iV of primary visual cortex contralateral to the stimulated eye. Power Diva equipment and software (Anthony Norcia; Smith Kettlewell Institute of Visual Sciences) was used for stimulus generation, data acquisition and analysis. While recording through one eye, the other eye was covered. Visual stimuli were presented on a CRT computer monitor and consisted of full-field sine-wave gratings at 80% contrast and reversing at 6.25 Hz. Sweep VEPs (sVEPs) were elicited by horizontally oriented gratings. The display was positioned 24 cm in front of the rat and centered at the vertical meridian. Mean luminance was held constant at 20 C/D. One stimulus presentation (one trial) consisted of a spatial frequency sweep decreasing from 1.6 to 0.03 cycles/degree in 15 linear steps. A total of 20 trials of 15 sec duration each are collected. Visual Acuity thresholds were estimated using Power Diva software. The same basic recording protocol was used to assess VEP power (ONC rats) except that the frequency was held constant throughout 10 trials of 15 sec each. Similarly, to assess contrast sensitivity, the protocol was the same except that the spatial frequency was fixed at 0.575 cpd throughout each trial while the contrast was swept from 2.5 to 70%. Sweep VEP recording sessions lasted between 10-20 min per animal.

What is claimed is:

1. A method of treating visual disorders mediated by the visual cortex by administering to a patient in need of such treatment, a therapeutically effective amount of a pharmaceutical composition comprising compounds acting at the alpha 2 adrenergic receptor.

2. The method of claim 1, wherein the composition comprises (5-bromo-quinoxalin-6-yl)-imidazolidin-2-ylidene-amine or a salt thereof.

3. The method of claim 1, wherein the visual disorder comprises amblyopia, stroke-induced blindness, visual system disorder in Parkinson's disease and Alzheimer's disease, seizure-induced cortical blindness, epileptic blindness, and induced visual system disorder including but not limited and to multiple sclerosis (MS)-induced visual system disorder, and congenital and childhood myotonic dystrophy type 1-induced visual system disorder.

4. The method of claim 1, wherein the visual disorder is optic neuritis.

5. The method of claim 1, wherein the visual disorder is amblyopia.

6. The method of claim 1, wherein the therapeutically effective amount of the pharmaceutical composition is administering topically.

7. An article of manufacture comprising packaging material and a pharmaceutical agent contained within said packaging material, wherein the pharmaceutical agent is therapeutically effective for lowering intraocular pressure and wherein the packaging material comprises a label which indicates the pharmaceutical agent can be used for lowering intraocular pressure and wherein said pharmaceutical agent comprises an effective amount of (5-bromo-quinoxalin-6-yl)-imidazolidin-2-ylidene-amine.
